(19) United States
(12) Patent Application Publication
(10) Pub. No.: US 2019/0029560 A1
(43) Pub. Date: Jan. 31, 2019

(54) Magnetic Marker for Surgical Localization

(71) Applicant: Endomagnetics Ltd., Cambridge (GB)

(72) Inventors: Quentin John Harmer, Cambridge (GB); Eric Mayes, Cambridge (GB); Quentin Andrew Pankhurst, Hertfordshire (GB); Andrew Shawcross, Cambridge (GB)

(73) Assignee: Endomagnetics Ltd., Cambridge (GB)

(21) Appl. No.: 15/867,165

(22) Filed: Jan. 10, 2018

Related U.S. Application Data
(63) Continuation of application No. 13/792,803, filed on Mar. 11, 2013, now abandoned.
(60) Provisional application No. 61/672,048, filed on Jul. 16, 2012.

(51) Int. Cl. A61B 5/06

(52) U.S. Cl. CPC A61B 5/062; A61B 5/4312

(57) ABSTRACT

A method and apparatus for preparing tissue of interest in a patient for possible excision by surgery. In one embodiment, the method comprises the steps of: removing a biopsy sample from the tissue of interest; placing a magnetic marker at the biopsy site; performing a pathology analysis of the biopsy sample; and if the pathology analysis indicates that the tissue of interest should be removed, locating the tissue for surgery using a magnetic detection probe. In one embodiment, the marker comprises magnetic nanoparticles in a bioabsorbable matrix. A system for preparing tissue of interest in a patient for possible excision by surgery. In one embodiment, the system includes a magnetic marker and magnetic detection probe system.

### CROSS-REFERENCE TO RELATED APPLICATION

This application claims priority to and the benefit of U.S. Provisional Patent Application No. 61/672,048, filed Jul. 16, 2012, the entire disclosure of which is hereby incorporated by reference herein, in its entirety and for all purposes.

### FIELD OF THE INVENTION

The invention relates in general to surgical devices and more specifically to devices that aid in locating a lesion for surgical excision.

### BACKGROUND OF THE INVENTION

With the increasing prevalence of mammography screening programs, the majority of breast cancers are detected as small, non-palpable (or occult) lesions, that are amenable to breast conserving treatment. Accurate localization of non-palpable breast cancers is key to allowing surgical removal of the complete tumor with adequate margins. If the tumor is not completely excised, patients need to undergo a further operation to remove any remaining cancerous tissue. Accurate localization also helps to avoid excision of excess breast tissue which could result in adverse cosmetic results.

FIG. 1 is a flow chart of current treatment algorithms for localization of non-palpable lesions. As a result of routine or screening mammography 10, a lesion or abnormality may be detected. If the abnormality is non-palpable, as part of further investigation, a tissue biopsy 14 is performed, typically using vacuum assisted biopsy (VAB) needle or core biopsy needle. Following the biopsy, an initial tissue marker is placed 16 so that the site can be located during subsequent follow up. The patient is then sent home 20 while the biopsy sample is analyzed. In the case that the sample is found to be benign, the tissue marker remains in the patient indefinitely 24. If the biopsy reveals a cancerous tumor, then the patient will return for surgery to remove the tumor.

The first marker cannot typically be located interoperatively because the imaging techniques used to locate it are not available during surgery; instead a further method of marking needs to be employed prior to surgery which can then be used interoperatively to assist the surgeon in localizing the lesion. The current gold standard for localization of non-palpable lesions during surgery is wire-guided localization (WGL). Shortly before surgery, a hook wire (or guidewire) is inserted 28 by the radiologist, guided by ultrasound or stereotactic x-ray imaging. During surgery, the surgeon follows the wire to its tip 32 to locate the lesion and removes the tissue surrounding the tip.

Although this technique is widely used, WGL has a number of disadvantages. First, it involves two separate procedures, and can present logistical and scheduling difficulties between radiology and surgery departments. Second, the positioning of the guidewire may not be optimal for achieving the desired cosmetic result in the subsequent surgery. Third, the hook wire can migrate away from the site of the lesion or become displaced during mammography or moving the patient. Fourth, the insertion of the wire can be painful for patients and finally, the risk of infection means that surgery usually needs to take place the same day as the wire insertion.

In order to overcome these disadvantages, other localization techniques have been developed. One such approach is Radioguided Occult Lesion Localization (ROLL). In this procedure, a radiotracer, typically a colloid labeled with Technecium-99 is injected 36 into the tumor site and a handheld gamma probe is used 40 by the surgeon to localize the tumor for excision. In a further development of ROLL, Radio Seed Localization (RSL), a titanium seed containing radioactive Iodine-125 is inserted into the tumor under X-ray or ultrasound guidance instead of a radiotracer injection.

These radioguided approaches require the logistical complexity of involving the nuclear medicine department as well as surgery, and introduce the drawback of the use of radioactive materials, which require special handling and disposal procedures 48. Some of the radioisotopes, for example Technecium-99, have short half lives, which constrains the time between administration and surgery.

A further disadvantage of all these approaches is that two types of marker need to be placed in the surgical site. First, after the biopsy, a tissue marker is placed to mark the biopsy site. Then at a later date, a further means of marking, either a guidewire, an injection of radiotracer, or a radioactive seed is placed in the same site. This duplication creates additional work for the surgical and radiology teams and inconvenience and increased risk for the patient.

Therefore, a need remains for a means for marking non-palpable lesions which avoids the drawbacks of the current techniques. The present invention addresses this need.

### SUMMARY OF THE INVENTION

In one aspect, the invention relates to a method of preparing tissue of interest in a patient for possible excision by surgery. In one embodiment, the method comprises the steps of: removing a biopsy sample from the tissue of interest; placing a magnetic marker at the biopsy site; performing a pathology analysis of the biopsy sample; and if the pathology analysis indicates that the tissue of interest should be removed, locating the tissue for surgery using a magnetic detection probe. In another embodiment, the marker comprises magnetic nanoparticles in a bioabsorbable matrix. In yet another embodiment, the removing of the biopsy sample comprises using an introducer and the placing of the magnetic marker at the biopsy site is performed through the introducer.

One aspect the invention relates to a magnetic marker. In one embodiment, the magnetic marker includes magnetic nanoparticles. In another embodiment, the magnetic marker includes magnetic nanoparticles and a bioabsorbable matrix. In another embodiment, the bioabsorbable matrix includes a bioabsorbable gel. In yet another embodiment, the bioabsorbable gel is expandable when in contact with tissue fluids.

In yet another aspect, the invention relates to a system for preparing tissue of interest in a patient for possible excision by surgery. In one embodiment, the system includes a magnetic marker and a magnetic detection probe system. In one embodiment, the magnetic marker is placed at the location of tissue removal after biopsy. In another embodiment, if excision of the tissue of interest is required, the magnetic detection probe is used to locate the position of the tissue of interest within the patient. In yet another embodiment, the magnetic marker comprises magnetic nanoparticles in a bioabsorbable matrix. In yet another embodiment, the magnetic detection probe system includes a magnetic probe; a power module in electrical communication with the magnetic probe to supply current to the magnetic probe; a sense module in electrical communication with the magnetic probe to receive signals from the magnetic probe; and a processing module in electrical communication with the power module and the sense module.

In one embodiment, the processing module generates a waveform that controls the supply of current from the power module to the magnetic probe. In another embodiment, the processing module receives a signal from the sense module that indicates the proximity to the magnetic nanoparticles. In still yet another embodiment, two magnetic probes are used, one for transcutaneous detection where a larger diameter probe can be used to detect to a greater detection depth, and a second smaller diameter probe for intraoperative detection where the probe diameter needs to be minimized to keep the incision size small and to facilitate visibility around the probe. In still yet another embodiment, the magnetic probe is constructed from a material having a coefficient of thermal expansion less than or equal to 10⁻⁵/° C. and a Young's modulus of substantially 50 GPa or more.

### BRIEF DESCRIPTION OF THE DRAWINGS

The objects and features of the invention can be better understood with reference to the drawings described below. The drawings are not necessarily drawn to scale; emphasis is instead being placed on illustrating the principles of the disclosed subject matter. The drawings associated with the disclosure are addressed on an individual basis within the disclosure as they are introduced.

FIG. 1 is a flow diagram of the marking methods known to the prior art;

FIG. 2 shows a treatment algorithm according to the present invention;

FIG. 3 shows the magnetic marker detection system;

FIG. 4a shows an embodiment of a magnetic marker in a hydrogel matrix and a biopsy needle placing the marker at the site of a lesion;

FIG. 4b shows an embodiment of a magnetic marker comprising gel spheres loaded with MNPs;

FIG. 5 shows an embodiment of a magnetic marker using beads filled with magnetic particles and a syringe placing the beads at the site of a lesion;

FIG. 6 shows an embodiment of a magnetic marker according to the invention comprising a bioabsorbable capsule filled with magnetic nanoparticles;

FIG. 7 shows an embodiment of a magnetic capsule containing magnetic nano-particles; and

FIG. 8 shows an embodiment of a compressible magnetic marker.

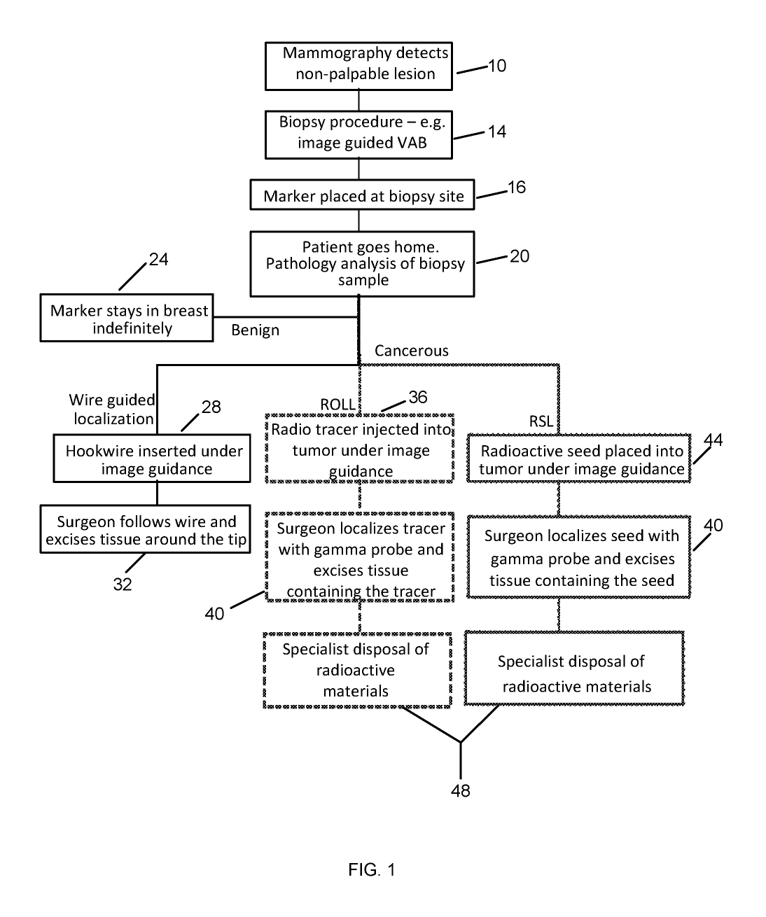

### DESCRIPTION OF THE ILLUSTRATIVE EMBODIMENTS

The present invention relates to a marker that can be positioned following a biopsy procedure, remains visible under a range of imaging modalities for a period of months in order to allow re-localization in cases where patients receive a course of neo-adjuvant chemotherapy before removal of the tumor, and is absorbed by the body after a number of months. In one embodiment, the marker is absorbed after 6 months.

The present invention discloses a marker for marking the site of the lesion with a magnetic material, for example a material containing superparamagnetic nanoparticles, which can be subsequently detected and localized interoperatively by using a handheld magnetometer. The marker may be placed at the site of the lesion at the time of the initial biopsy. In the case where the biopsy sample indicates that the lesion is benign, the marker remains in the breast and is absorbed over a period of time, in one embodiment, six months.

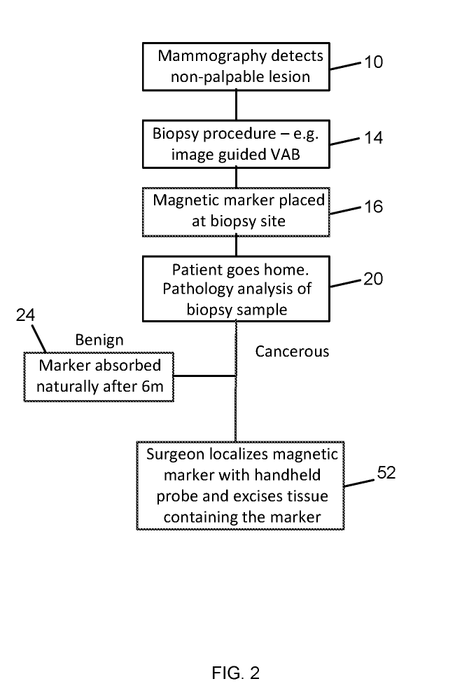

If the biopsy shows that the lesion is cancerous, then the marker is used to identify the site of the lesion. The marker is preferably visible under usual imaging modalities such as MRI, ultrasound and X-ray for pre-operative imaging, if required. During surgery, the surgeon can use a handheld magnetometer such as that disclosed in U.S. patent application Ser. No. 12/631,370 filed Dec. 4, 2009 and Ser. No. 12/960,746 filed Dec. 6, 2010, the full specification of each are herein incorporated by reference, to localize 52 (FIG. 2) the marker interoperatively in order to excise the lesion. Using this approach, only a single marker needs to be placed, no guidewire is required, and the use of radioactive materials is avoided. This reduces the number of procedures that the patient has to undergo, reduces the amount of work for the surgery and radiography teams, and reduces the resources used and therefore the overall cost of removing the lesion. Note that if required, the marker can also be used to guide the insertion of a guidewire to allow a conventional WGL procedure if the magnetic probe is not available.

If the surgeon wants to mark the margins of a tumor, more than one marker may be placed. In this case, preferably the markers have form or features to distinguish one marker from the others so that the orientation of the tumor can be identified once it is removed to allow correct identification of any regions where further tissue removal is needed, and thus to ensure that all the tumor has been removed with sufficient margin around it. A magnetic marker may also be placed at the site of small tumors or micrometastases prior to neo-adjuvant therapy so that the effect of the therapy can be monitored.

In one embodiment, the marker includes magnetic nanoparticles (MNPs). MNPs are known for use as magnetic markers for sentinel lymph node localization. When sufficiently small, these particles exhibit superparamagnetic behavior whereby they become magnetized in the presence of a magnetic field and exhibit no permanent magnetic remanence when the field is removed. This property can be used to allow the detection and localization of the particles using a sensitive magnetometer (or susceptometer) which generates an alternating magnetic field to excite the particles magnetically, and detects the magnetic field signature generated by the particles. Such a device is described in each of Ser. No. 12/631,370 and Ser. No. 12/960,746.

The particles typically contain an iron oxide (magnetite and/or maghaemite) core surrounded by a biocompatible coating such as dextran, carboxydextran, other sugars, albumin, PEG, or biocompatible polymers. To exhibit superparamagnetic behavior, the particles' magnetic cores need to be below a critical diameter, typically in the range 3-25 nm depending on the material and structure.

As well as coating to enhance biocompatibility, MNPs are often coated in order reduce toxicity, prevent agglomeration of the particles, or to modify their residence time in the body. Coating materials are typically natural or synthetic polymers including dextrans, carboxydextrans, Poly ethylene glycol (PEG), Poly vinyl alcohol (PVA), polyvinylpyrrolidone (PVP), polyethyleneimine (PEI), polyglucose sorbitol carboxymethylether and chitosan. Other coating materials include metals such as gold, pegylated colloidal gold nanoparticles, silver; carbon, silica, silicones, aminosilanes and ceramics.

MNPs can also be functionalized to allow them to localize in particular tissue or cell types, for example cancerous cells, or to target particular biological systems in order to deliver therapies to those areas. Functionalizing is achieved by attaching or coating with biovectors comprising for example antibodies, enzymes or proteins.

In one embodiment, iron oxide is used for the superparamagnetic core because of its low toxicity, but other materials which can form a superparamagnetic core are acceptable. The material of the core should be one that is capable of being magnetically ordered. It may be a metal, such as cobalt, iron, or nickel, a metal alloy, rare earth and transition metal alloy, M-type or spinel ferrite containing aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium, or a mixture thereof The core can also be formed by oxidizing a combination of an iron (II) salt and another metal salt. The metal salts which are beneficial include salts of aluminum, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium.

MNPs for sentinel node location as described in each of Ser. No. 12/631,370 and Ser. No. 12/960,746 are sized so that they are taken up into the lymphatic system and flow to the lymph nodes where they are trapped by virtue of their size. This means that they need to have a hydrodynamic diameter in the size range 5-200 nm typically, and preferably in the range 20-150 nm. Particles of this size may be suitable for marking a biopsy site but a proportion will also tend to migrate into the lymphatic system.

While MNPs are the preferred type of magnetic marker, a sensitive magnetometer of the kind disclosed in the applications referenced above is equally able to detect other ferromagnetic materials, and even conductive materials, although the strength of signal for both is typically lower than for an equivalent mass of MNPs. Thus, other forms of magnetic marker constructed from any kind of ferromagnetic or conductive material including soft iron, ferrites, stainless steels, titanium, nickel and related alloys can be envisaged within the scope of the invention. These may, for example, be in the form of a plug of material or beads, microspheres or particles formed from the material. In order to ensure biocompatibility, these materials may be coated with a biocompatible or inert material, for example titanium, gold or carbon.

Other forms of magnetic sensing technology may be envisaged to detect and localise the marker including magnetostrictive, hall effect, SQUID based, fibre optic, magneto-optical and alternative search coil sensors.

Surgical devices that are absorbed after they have achieved their function, for example absorbable sutures, are well known in the art. By choice of the correct combination of materials (typically bioabsorbable polymers), the time over which the device is absorbed can be controlled from a few weeks to several months. For magnetic tissue, marking the ideal period over which the marker is absorbed depends on the application. For temporary applications it is desirable for the marker to be absorbed in only a few weeks, e.g. 1-4 weeks, while more typically, where the marker may need to remain in place until a course of neo-adjuvant therapy has been completed, the ideal absorption time is a number of months, e.g. 1, 2, 3 or 6 months. Exceptionally, a marker may need to remain in-situ for longer than six months, for example in the case where a suspicious lesion is presumed benign, but needs to be monitored for 6, 9, or even 12 months.

In one embodiment of the present invention, a tissue marker is provided comprising superparamagnetic MNPs loaded into an expanding bioabsorbable gel matrix or other means for immobilizing them. Upon insertion following breast biopsy, the marker expands as it absorbs water from the surrounding tissue such that it fills the cavity left when the biopsy sample has been removed. The MNPs are held in the gel or other matrix. Upon excitation by an alternating magnetic field, the MNPs emit a magnetic field, which can be detected by a sensitive magnetometer. The marker preferably remains magnetically traceable for a period of time for example 1, 2, 3 or 6 months after which time the bioabsorbable material and the MNPs it contains are absorbed. In a further aspect, the bioabsorbable gel in which the MNPs are loaded is in the form of small spheres, which can be injected. Following injection, the spheres absorb water and expand to fill the biopsy cavity. Preferably they also fuse with each other to form a single plug to prevent migration of the marker.

In another embodiment of the present invention, a tissue marker is provided comprising bioabsorbable polymer beads loaded with superparamagnetic MNPs. The beads can, for example, be inserted during a breast biopsy. Preferably, the beads fuse together once introduced to form a plug. The magnetically traceable beads mark the site for subsequent surgery. The marker remains magnetically traceable for a period of time after which it is absorbed.

In another embodiment of the present invention, a magnetic tissue marker is provided comprising two or more liquid components including MNPs which are mixed just prior to or at the time of injection. Upon injection, the components react to form a plug in-situ, trapping the MNPs. The components may be, for example, the components of a copolymer that combine to form a cross-linked polymer. The material is injected during a breast biopsy to form a magnetically traceable plug which marks the site for subsequent surgery. The marker is absorbed after a period of time.

In another embodiment of the present invention, a tissue marker is provided comprising a concentration of superparamagnetic MNPs loaded into a sealed seed or pellet formed from a bioabsorbable material. The marker can be introduced during a breast biopsy procedure and magnetically marks the site for surgery. The marker is preferably absorbed after a period of time as the seed material is absorbed.

In another embodiment of the present invention, a tissue marker is provided comprising superparamagnetic MNPs individually coated in a bioabsorbable material. The particle size is chosen such that the particles do not migrate through the tissue or get taken up in the lymphatic system. The particles can be suspended in a biocompatible liquid for example saline or water for injection, and injected at the time of a breast biopsy to magnetically mark the site of the lesion. The marker is absorbed after a period of time as the absorbable coating material is broken down.

In another embodiment of the present invention, a marker is provided that comprises MNPs with two or more size populations, for example a population with a mean particle size of around 60 nm for uptake into the lymphatic system for sentinel node detection or mapping, and a population with mean particle size greater than 500 nm for tissue marking. This marker could be delivered to a tumor or biopsy site to both mark the lesion and to map out the sentinel lymph nodes prior to a magnetic SLN detection. The particles, particularly the larger size population, may be coated with a bioabsorbable material to increase their residence time in the tissue.

In another embodiment of the present invention, the magnetic marker also contains some starch or other haemostatic material to assist with haemostasis following a biopsy.

A further advantage of the present invention is that iron oxide magnetic nanoparticles are visible by most of the most common medical imaging methods. Iron oxide MNPS are known in the art to be visible under MRI and they have been used as contrast enhancement agents. MNPs also are visible under the various X-ray based imaging modalities. A number of imaging techniques are under development which offer the possibility of real-time imaging in the operating room during surgery; for example, photo-acoustic imaging, magnetic particle imaging, magneto-acoustic imaging, and magneto-photo-acoustic imaging. Iron oxide MNPs are also potentially visible using all these imaging techniques.

To enhance the visibility of the marker under X-ray, an additional X-ray visible element may be added to the marker. This may be a solid metal element, for example, a small piece of titanium or stainless steel or an X-ray visible ceramic such as zirconia. Various forms and shapes are suitable including clips, springs, coils, wires, cylinders, rings and elements formed from sheet material. The element may be embedded in the marker or delivered with the marker. Preferably, the element is associated with the marker so that it marks the desired site and is not subject to migration. Where appropriate, the element is coated to enhance biocompatibility.

X-ray visibility can also be achieved by the addition of radiopaque compounds, for example compounds containing barium or iodine compounds, or other heavy elements. This approach is advantageous for liquid or gel markers as the radiopaque compounds can also be formulated in this form.

Iron oxide MNPs are not typically visible using ultrasound and therefore the other components of the magnetic marker are preferably chosen such that the marker can be seen using ultrasound. For example, some hydrogels can be visible under ultrasound.

In a further embodiment of the present invention, a method for localizing occult (non-palpable) lesions is provided, including the steps of marking the site of the lesion following removal of a breast biopsy sample with a marker containing magnetically detectable material; detecting and localizing the marker interoperatively during surgery by using a handheld magnetometer; and excising the lesion containing the marker.

In more detail, FIG. 2 is a flow chart of a treatment algorithm for non-palpable lesions according to the present invention. Once an abnormality or tumor is identified during routine mammography 10, further follow up is carried out. If the lesion is non-palpable, a biopsy is carried out 14 to remove a sample of the lesion for analysis. Following the biopsy, a magnetic marker is placed 16 to mark the site of the lesion and the patient is sent home. In the case where the biopsy sample indicates that the lesion is benign, the marker remains in the breast and is absorbed over a period of time 24, for example, six months. If the biopsy shows that the lesion is cancerous, then the marker is used to identify the site of the lesion. The marker is visible under usual imaging modalities such as MRI, ultrasound, and X-ray for pre-operative imaging if required. During surgery, the surgeon uses a handheld magnetometer (e.g. as disclosed in the application noted above) to localize the marker 52 interoperatively and excise the lesion.

Only a single marker is placed at the time of the initial biopsy, and the use of a second marker, guidewire, or radioactive source is eliminated. This reduces the number of procedures that the patient has to undergo, reduces the amount of work for the surgery and radiography teams, and reduces the resources used and therefore the overall cost of removing the lesion. Furthermore, the use of radioactive materials is not required. Because the marker remains in position for a period of time, it is suitable for use when patients undergo a course of neo-adjuvant chemotherapy before removal of the lesion.

Whilst FIG. 2 outlines an embodiment of the method of use, other algorithms using the magnetic marker can be envisaged and fall within the scope of the invention. For example, if required, the marker can also be used to guide the insertion of a guidewire or other localization method to allow a conventional WGL procedure if the magnetic probe is not available. In a further instance, the magnetic marker is palpable and can be located by palpation.

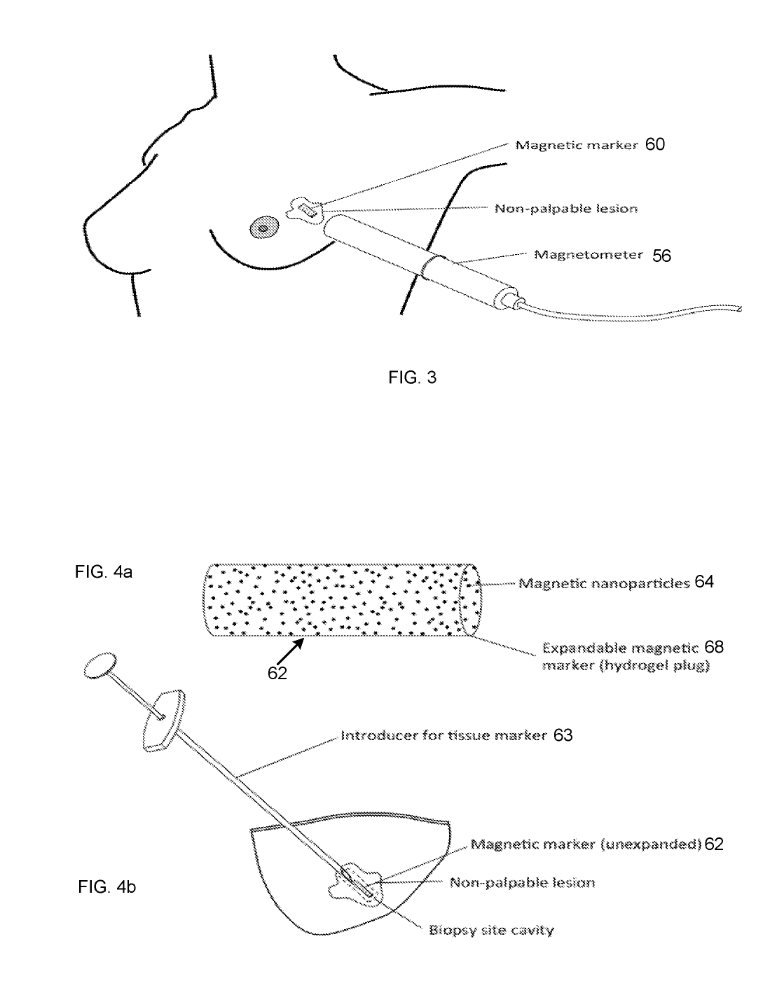

FIG. 3 shows a magnetic marker according to the current invention being detected by a magnetic marker detection system. When the lesion needs to be removed, during the procedure, the surgeon can use a magnetometer 56 (susceptometer) of the kind disclosed in the above-referenced applications, to localize the marker 60. This would happen typically in two stages: 1) the magnetometer is used transcutaneously prior to making an incision to locate a ‘hotspot’ indicating the region of the breast in which the marker is located and suggesting a site for an incision; and 2) once an incision has been made, the magnetometer can be used interoperatively in the incision to localize the marker and thereby localize the lesion to be removed. The lesion is then removed along with a margin around it and the marker contained within the lesion. The magnetic marking and localization method is not limited in its application to breast cancer lesions and can be applied in any indication where marking of a surgical site is needed.

FIG. 4a shows an embodiment of a magnetic marker 62 according to the current invention, and an introducer 63 being used to place the marker at the site of the lesion. The marker 62 comprises superparamagnetic MNPs 64 loaded into an expanding bioabsorbable gel matrix 68. The introducer is inserted into the breast tissue following the biopsy and the plunger is depressed to insert the marker into the cavity left by the removal of the biopsy sample. Upon insertion, the marker expands as it absorbs water from the surrounding tissue such that it fills the biopsy cavity. The MNPs are held in the gel or other matrix. Upon excitation by an alternating magnetic field, the MNPs emit a magnetic field which can be detected by a sensitive magnetometer. The marker preferably remains magnetically traceable for a period of time, for example 1, 2, 3 or more preferably 6 months after which time the bioabsorbable material and the MNPs it contains are harmlessly absorbed by the body, primarily through uptake in the reticuloendothelial system, and the absorbed iron becomes part of the body's iron stores.

The unexpanded marker is sized so that it fits within an introducer for a tissue marker, typically less than 2.4 mm in diameter for a vacuum assisted biopsy system, or 1.4 or 0.8 mm diameter for smaller gauge needle biopsies. Once in-situ, the marker expands to fill the cavity left by the biopsy. The volume of the marker increases by a factor of at least 3 and preferably more than 5. The length of the marker is sized to fit into an introducer and is less than 15 mm, and more preferably between 3 and 10 mm.

In a further aspect of this embodiment (FIG. 4b), the bioabsorbable expandable gel in which the MNPs are loaded or embedded is in the form of small spheres in a dry or non-hydrated state. The spheres are mixed with water or other suitable fluid immediately prior to injection. Following injection with the water, the spheres absorb water and expand to fill the biopsy cavity. Preferably, they also fuse with each other to form a single plug to prevent migration of the marker. The spheres are small enough to be injected, i.e. less than 0.8 mm and preferably less than 0.4 mm in diameter.

The matrix may be made from a suitable expandable bioabsorbable material. Examples of bioabsorbable materials that expand when in the presence of aqueous fluids such as biological fluids include hydrogels, collagen, and other suitable hydrophilic materials. Biodegradable hydrogels can be prepared by incorporating one or more of the following monomers: glycolide, L-lactide and its isomers, ε-caprolactone, p-dioxanone and trimethylenecarbonate (TMC) within what otherwise be considered non-biodegradable polymer hydrogels.

Examples of suitable classes of polymer hydrogels include those formed from one or more of the following monomers: hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, ethylene glycol dimethacrylate, N-vinyl-2-pyrrolidone, N-isopropyl AAm, vinyl acetate, Acrylic acid, MAA, N-(2-hydroxypropyl) methacrylamide, ethylene glycol, PEG acrylate, PEG methacrylate, PEG diacrylate, PEG dimethacrylate. In addition, biodegradable hydrogels can be based on natural products such as dextrans, beta-glucan, silk fibroin or polypeptides like gelatin which may be cross-linked with aldehydes such as formaldehyde or glutaraldehyde.

Advantageously, the marker material is chosen such that the expanded marker is harder than the surrounding tissue, and preferably significantly harder, so that a surgeon can locate the lesion by palpation.

The marker needs to contain sufficient magnetic material to be detectable externally using a magnetic probe. For example, for a breast lesion, the marker needs to be detectable from at least 25 mm and preferably 40 mm or more. In order to achieve this, the marker needs to contain sufficient mass of iron oxide particles, for example at least 1 to 2.5 mg of iron oxide, preferably more than 5 mg and more preferably more than 10 mg or even 20 mg. It is therefore beneficial for the density of iron oxide particles within the marker to be as high as possible, i.e. greater than 10 mg/cm³ of marker, and preferably greater than 20 mg/cm³, and more preferably greater than 50 mg/cm³.

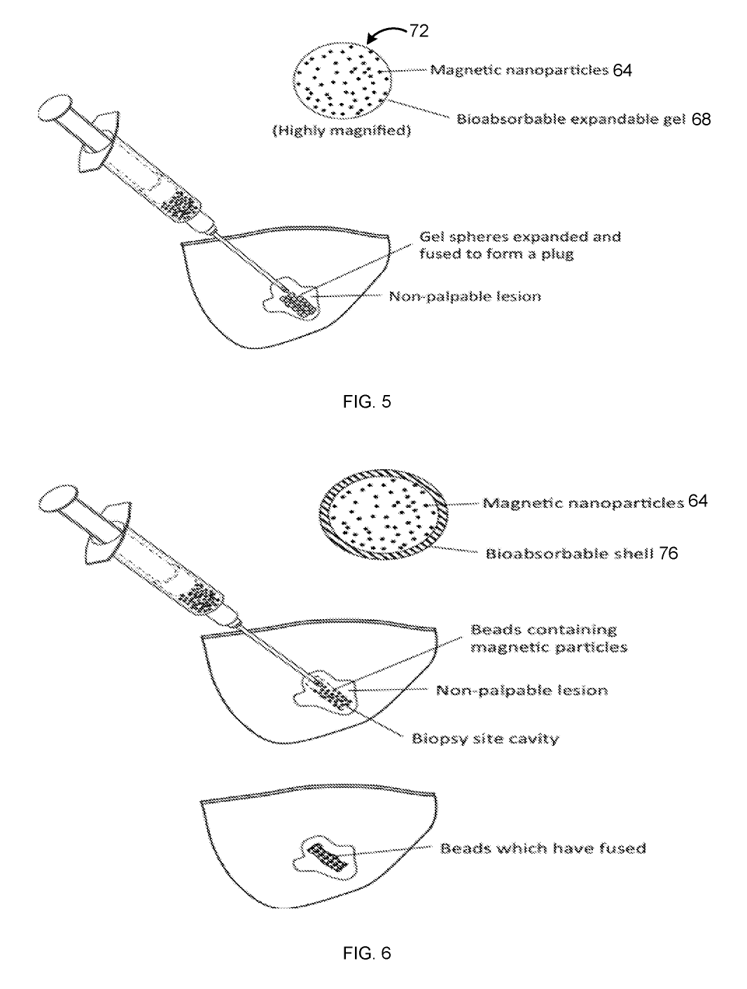

FIG. 5 shows an embodiment of a bioabsorbable polymer bead 72 or sphere filled with superparamagnetic MNPs 64, and a quantity of the beads being injected into a biopsy site to magnetically mark the site of a non-palpable lesion. FIG. 5 further shows the beads subsequently fused together to form a magnetic marker plug.

The beads comprise a matrix 68 or shell of a bioabsorbable polymer filled with magnetic particles 64. The particles 64 may be suspended in a liquid or gel, for example water. Alternatively, the particles can be encapsulated in a bioabsorbable polymer 76. Such encapsulation techniques are known for creating microspheres for controlled-release drug delivery. The beads can, for example, be inserted during a breast biopsy. The magnetically traceable beads mark the site for subsequent surgery. The marker remains magnetically traceable for a period of time for example 1, 2, 3 or more preferably 6 months, after which it is absorbed along with the MNPs.

The beads can be formed from any of a number of suitable bioabsorbable polymers. Examples of suitable natural bioabsorbable materials include collagen, gelatin, and other cellulose base materials. Examples of suitable synthetic bioabsorbable materials include hydrogels, Polyvinyl alcohol (PVA), and Polyglyconate. Also synthetic bioabsorbable polyester-based materials formed by homopolymerization or copolymerization of one or more of these monomers: glycolide, L-lactide and its isomers, ε-caprolactone, p-dioxanone and trimethylenecarbonate (TMC). These may include homopolymers such as Poly(L-lactide) Poly(DL-lactide), Poly(TMC), Polycaprolactone (PCL), Polyglycolide (PGA), Poly(glycolide-L-lactide) (PGL), or Poly(p-dioxanone) (PDS); or co-polymers such as: L-Lactide/DL-Lactide, L-lactide/Glycolide, L-lactide/Caprolactone, DL-Lactide/Glycolide, DL-Lactide/Caprolactone, Glycolide/Caprolactone, L-lactide/Glycolide/Caprolactone, DL-Lactide/Glycolide/Caprolactone, Poly(dioxinone co-trim ethylene carbonate-co-glycolide) Glykomer 631 (marketed as Biosyn®); or copolymers of these with PDS.

Preferably, the beads fuse or adhere to each other once at the site of the lesion to form a cohesive plug so that the beads do not migrate away from the site to be marked. The adhesion may be achieved by means of a coating or surface property of the material, which becomes adhesive on contact with aqueous fluids or other biological fluids. The fluid may for example be mixed with the beads just prior to injection. Alternatively, the fusing may be achieved by the combination of components of a copolymer to form a cross-linked polymer.

The beads are sized to flow through a conventional biopsy needle or introducer. Conventional biopsy needles are 14, 16, 18 gauge needles (inner diameter 0.8-1.4 mm), while a vacuum-assisted biopsy needle is 11 gauge (2.4 mm inner diameter).

It may be desirable to deliver the beads via a conventional hypodermic needle which is typically 21 to 33 gauge. Thus, the beads are less than 0.5 mm, preferably less than 100 μm, and more preferably in the range 10-50 μm in diameter to allow delivery via a needle. Particles less than 10 μm in diameter are more likely to be taken up by macrophages and therefore absorbed prematurely, which is undesirable. Preferably, the beads have a narrow particle size distribution with a coefficient of variation in the mean diameter of less than 10%, and ideally less than 5% so that they behave in a more uniform manner.

Needle delivery of beads may be desirable in the case where the surgeon wishes to mark the tumor margin in multiple places, because using the needle, a small amount of beads can be placed in several locations around the tumor.

FIG. 6 shows a magnetic marker comprising a bioabsorbable seed or pellet filled with magnetic nanoparticles 64. The particles may be suspended in a liquid or gel, for example water. Alternatively (not shown) the pellet comprises a matrix of the bioabsorbable polymer loaded with MNPs. Suitable bioabsorbable materials include those listed above.

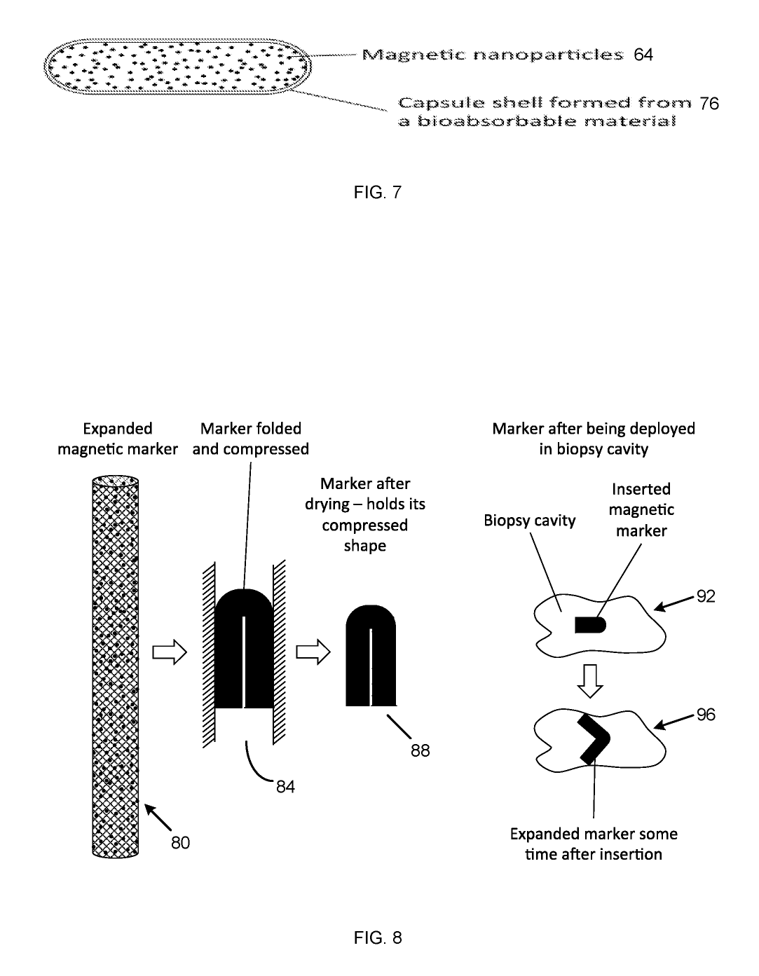

The seed is sized so that it fits within an introducer for a tissue marker, typically less than 2.4 mm in diameter, and more preferably less than 0.8 mm diameter. The length of the marker is sized to fit into an introducer and is less than 15 mm, and more preferably between 3 and 10 mm (FIG. 7).

The pellet can, for example, be inserted during a breast biopsy. The pellet is magnetically traceable and marks the site for subsequent surgery. The bioabsorbable material allows the pellet to remain magnetically traceable for a period of 1, 2, 3 or more preferably 6 months, after which it is absorbed.

FIG. 8 shows a magnetic marker 80 comprising an absorbable compressible or sponge-like material loaded with MNPs 64 that can be dried or desiccated. The material is subject to a configuration change such as folding 84, and then compressed and dried prior to delivery, e.g. during manufacture, such that the marker is several times smaller than its original size. By virtue of drying, the material is able to hold its compressed shape 88 until it is rehydrated on deployment 92. On deployment, as it absorbs water, it returns to its original shape 96 and in so doing, anchors itself securely in the biopsy cavity. Preferably, the marker is also swellable such that on drying it becomes smaller and then expands again on rehydration. This enables the change in volume between the pre-deployed and post-deployed states to be maximised. The change in volume is at least 4 times and preferably 5 to 15 times the original volume.

Suitable materials for the marker include the hydrogel materials listed previously. Various shapes and configurations for the marker can be envisaged, including cylindrical, spiral, helical shapes; and zig-zag, concertina, unfolding and untwisting configurations. Compressible net and mesh configurations are also suitable.

In a further embodiment of the invention (not shown), a magnetic marker is provided comprising a liquid or gel injection including MNPs. The material may be sensitive to the presence of a biological fluid or biological condition such as body temperature or pH such that it forms a gel or solidifies in-situ in the body. The material is injected during a breast biopsy to form a magnetically traceable marker that marks the site for subsequent surgery. Suitable materials include Hyaluronic acid, Polyethylene glycol, Dextran and collagen. A liquid solution of collagen may be configured to solidify on injection by a change of pH once in the biopsy cavity. The volume of the injection may be in the range of 0.1 to 5 ml and more preferably 0.2 to 1 ml.

In a further embodiment of the invention (not shown), a magnetic marker comprises two or more liquid components including MNPs which are mixed just prior to or at the time of injection. Upon injection, the components react to form a bioabsorbable plug in-situ, trapping the MNPs. The components may be sensitive to the presence of biological fluid such that they form a solid or gel on contact with them, or they may be heat sensitive such that they form a solid or gel on exposure to body temperature. Preferably, the components are the components of a copolymer that combine to form a cross-linked polymer. The material is injected during a breast biopsy to form a magnetically traceable plug which marks the site for subsequent surgery. The bioabsorbable material allows the marker to remain magnetically traceable for a period of 1, 2, 3 or more preferably 6 months, after which it is absorbed.

In a further embodiment of the invention (not shown), a tissue marker is provided comprising superparamagnetic MNPs individually coated in a bioabsorbable material. The particle size may be chosen such that the particles do not migrate through the tissue or get taken up in the lymphatic system during the life of the marker. The particles are greater than 50 nm in diameter, preferably greater than 200 nm in diameter, and more preferably greater than 500 nm in diameter. The particles can be suspended in a biocompatible liquid, for example saline or water for injection, and injected at the time of a breast biopsy to magnetically mark the site of the lesion. The bioabsorbable material allows the marker to remain magnetically traceable for a period of 1, 2, 3 or more preferably 6 months, after which it is absorbed. The bioabsorbable coating may comprise any of the polymer materials, or any of the natural materials, or any of the biocompatible coating materials (dextran etc.) mentioned above.

In a further embodiment of the invention (not shown), a tissue marker is provided comprising superparamagnetic MNPs with two or more size populations, for example a population with mean particle size of around 60 nm for uptake into the sentinel lymph node for sentinel lymph node (SLN) detection and a population with a larger particle size suitable for tissue marking. Preferable particle sizes for SLN detection are in the region of 20 to 150 nm and particle sizes for marking are greater than 150 nm and preferably greater than 200 nm.

The use of magnetic nanoparticles in cancer is also known for:

1) Delivering drug and biological therapies to tumors, for example by attaching a pharmacologically or biologically active agent to the nanoparticles and delivering the particles to the tumor site.

2) Hyperthermia treatment whereby nanoparticles are concentrated at the tumor site and a high power alternating magnetic field is applied to the particles which heats them up and kills the surrounding tumor cells.

It is clear that the magnetic marker of the present invention could be used in combination with either of these treatment techniques to deliver therapy to the tumor as well as marking its position.

It is to be understood that the figures and descriptions of the invention have been simplified to illustrate elements that are relevant for a clear understanding of the invention. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the claims.

Variations, modification, and other implementations of what is described herein will occur to those of ordinary skill in the art without departing from the spirit and scope of the invention as claimed. Accordingly, the invention is to be defined not by the preceding illustrative description, but instead by the spirit and scope of the following claims.

What is claimed is:

1-11. (canceled)

12. A magnetic marker comprising:
a single plug made of a single magnetically detectable ferromagnetic material,
wherein the magnetic marker is between 0.8 mm and 2.4 mm in diameter and less than 15 mm in length and
wherein the magnetic marker has magnetic susceptibility such that the magnetic marker is detectable using a handheld magnetic susceptometry probe.

13. The magnetic marker of claim 12 wherein the ferromagnetic material is a stainless steel comprising at least 1 mg. of ferromagnetic material.

14. The magnetic marker of claim 12 wherein the plug of magnetic material is detectable by one or more of X-ray, ultrasound, magnetometer and MRI.

15. The magnetic marker of claim 12 wherein the marker is implantable for less than 6 months.

16. The magnetic marker of claim 12 wherein the marker is between 3 and 10 mm in length.
